(19) United States
(12) Patent Application Publication
(10) Pub. No.: US 2012/0217317 A1
(43) Pub. Date: Aug. 30, 2012

(54) Seed Mixture and Method of Application

(76) Inventor: Troy A. Hake, Independence, OR (US)

(21) Appl. No.: 13/036,355

(22) Filed: Feb. 28, 2011

(51) Int. Cl. A01C 15/02; A01H 5/00; A01C 7/06; A01C 5/02; A01G 25/09

(52) U.S. Cl. 239/1; 800/298; 111/95; 239/652

(57) ABSTRACT

A novel groundcover seed mixture and method of application comprising mixing visual powdered indicator with small groundcover plant seeds thus allowing a more even and visible application of the groundcover plant seeds over the desired ground surface area.

### FIELD OF INVENTION

This invention relates generally to a seed mixture, and more particularly to an improved mixture and method of dispensing small groundcover seeds.

### BACKGROUND OF THE INVENTION

Historically, planting small seeds was a labor intensive job. This is especially true for groundcover seeds that are traditionally dispensed by hand. More often then not, the seeds were not spaced properly thus requiring time consuming labor of thinning the plants after the seeds have sprouted. Furthermore, due to the minuscule size of most groundcover plant seeds such as Irish moss (40,000-60,000 seeds per gram), the difficulties of properly spreading the seeds multiply due to the difficulty in handling such minute volumes of seeding material and the inability to see the seeds during and after the spreading. As well, often the seeds in question are very expensive making inefficient application particularly wasteful.

A variety of devices and methods are known for spreading material, such as, grass seed, fertilizer, and various granular and powdered materials. For example, U.S. Pat. No. 1,274,969 by Wirt discloses a broadcast sower for distributing seed from a container in a plurality of different directions. U.S. Pat. No. 2,511,560 by Bechmann discloses a scoop for manually scattering powdered or granular materials, such as, lime, fertilizer, or seeds with the scoop provided with a plurality of vanes to direct the material outwardly from the scoop. U.S. Pat. No. 4,998,674 by Torra discloses a telescopic nozzle to uniformly distribute granular material. Although these prior art are capable in their own right, they fail to independently address seed sizes that are both small in volume and weight and near impossible to see during and after the spreading of the seeds.

Other prior art such as U.S. Pat. No. 5,664,506 by Morin focuses on a device and method for dispensing smaller seeds. Morin teaches of an electronic hand held dispenser that is designed to enhance regularity in the dispensing of very small seeds. However, Morin fails to address the need for the user to visually see the locations of where the small seeds have been spread. Furthermore, the singular application of very small seeds is not practical for groundcover plant species which require the even distribution of the groundcover seeds over a large ground surface area.

U.S. Pat. No. 6,283,051 by Yoss also attempts to solve the difficulties in spreading smaller seeds by disclosing a device in which a movable seed carrier plate containing seed receptacles carries seed receptacles to first and second positions. In the first position, a vacuum source draws seeds from the seed reservoir into the receptacles to a second position where a pressure source pushes the seeds from the seed receptacles. Although Yoss focuses on seeds of smaller sizes, it is overly complicated and fails to address the need to visualize the application of the smaller seeds. Furthermore, Yoss would not be practical in spreading groundcover seeds due to its dependency on planters tape.

Therefore, the prior art are not well suited for dispensing small groundcover seeds such as Irish moss and other species of groundcover plant seeds. The prior art devices rely on overly complicated mechanisms that fail to address the need to visually see and indicate the locations where the groundcover seeds have been dispensed. It is further clear that small seeds may be so light that the weight of the seed itself is not enough to dislodge the seed from the traditional seed carrier and dispensers. Moreover, the weight of the seed may not be enough to force the seed through the dispensing tube, thereby leading to blockage of the dispenser. Still further, the devices of the type described above tend to dispense seeds en masse, which further frustrates the purpose of spreading groundcover seeds evenly and efficiently.

Thus, it can be appreciated that there is a need in the art for a method which can dispense very small groundcover seeds accurately and efficiently without seed clumping and with a visible indicator to indicate areas seeded. These and other objects and advantages of the present invention will be more readily apparent from a consideration of the following drawings and a detailed description of the preferred embodiment.

### SUMMARY

It is therefore an object of the present invention to overcome the aforementioned disadvantages of prior art devices and provide a means to properly distribute groundcover seeds. The present invention teaches a groundcover seed mixture that comprises of a majority percentage by total volume of a powdered indicator mixed with a minority percentage by total volume of groundcover plant seeds. The powdered indicator may comprise of powdered lime which is white in color.

The method of dispensing the groundcover seed mixture comprises the steps of combining the majority percentage by total volume of the powdered indicator with the minority percentage by total volume of the groundcover plant seeds within a portable seed dispenser. The portable seed dispenser may be shaken to evenly mix the powdered indicator with the groundcover plant seeds. With the proper ratio between powdered indicator and groundcover plant seeds, only a light dusting of the mixture is needed over the desired planting location. The powdered indicator allows the visualization of the light dusting and allows visualization of the locations with prior applications. The powdered indicator further alleviates the problems of distributing the minuscule seeds by themselves by providing overall volume easing material handling. These and other novel features and advantages of the invention will be described in greater detail in the following detailed description.

### BRIEF DESCRIPTION OF THE DRAWINGS

The above description and other objects, advantages, and features of the present embodiment will be more fully understood and appreciated by reference to the specification and accompanying drawings, wherein:

FIG. 1 is a perspective view of the portable seed dispenser containing a mixture of powdered indicator and groundcover seed.

### DETAILED DISCUSSION OF THE PREFERRED EMBODIMENTS

Groundcover plants generally comprise low growing, typically under ten inches in height, species such as: Irish Moss, Alyssum, Chamomile, Lobelia, Moss Rose, Creeping Thyme, Ice Plant, Prunella, Aubrieta, Saxifraga, Leontopodium, Erigeron, Cerastium, Creeping Speedwell, Cymbalaria, & Sedum. Groundcover plant seeds are generally smaller than grass and vegetation seeds and tend to grow more horizontally than vertically. The application of groundcover seeds is different due to the seed size, price per gram, and limited areas of application. For example, 1,000 square feet lawn might require ten pounds of grass seed for coverage. However, to cover the same 1,000 square feet area with Irish moss, only 50,000 Irish moss seeds would be required, which is approximately one tea spoon worth of seeds, making it very difficult to spread such a small volume over such a broad area. Although more seeds may be used, it would not be economical due to the high cost of groundcover seed. Ten pounds of many species of groundcover plant seeds would cost thousands of dollars. Furthermore, over seeding would lead to waste, over competition and would ultimately be detrimental to the entire process.

Groundcover seeds are often planted in hard to reach areas such as rock gardens or in between stepping stones. Traditional methods of broadcasting seed wildly over the top of the area would be ineffective, wasting many of the expensive seeds. A controlled method of application is needed where the proper amount of seeds must be precisely sprinkled over the required area. The same is true for rock gardens where other plant species already exist. Traditional dispensing seeds in a broadcast manner would put seeds in places where it is not desired.

In the preferred embodiment of the present invention, a plastic bottle seed dispenser 10 with a detachable pores cap 12 is used to house a mixture of powdered indicator 14 and groundcover plant seeds 16. In the preferred embodiment, white powdered lime (calcium hydroxide) is used as the powdered indicator and is combined with Irish moss seeds wherein the seed size is approximately 40,000-60,000 seeds per gram. The operable mixture ratio is 98-99.99 percent by total volume of the powdered lime mixed with 0.01-2 percent by total volume Irish moss seeds. The preferred mixture ratio is 99.01-99.99 percent by total volume of powdered lime mixed with 0.01-0.99 percent by total volume of Irish moss seeds. The optimal mixture ration is 99.51-99.99 percent powdered lime mixed with 0.01-0.49 percent by total volume Irish moss seeds.

In the optimal ratio, a ten oz bottle seed dispenser contains approximately 10,000 Irish moss seeds, approximately 9.95 oz of powdered lime and can cover 200 square feet of ground surface. The bottle seed dispenser 10 is portable and can be shaken manually both vertically and horizontally to evenly blend the powdered lime and the Irish moss seeds. In most circumstances, shaking the mixture for twenty seconds will provide an even dispersion of the Irish moss seeds within the powdered lime creating a fluidized mixture. Once the mixing is complete, the mixture may be dusted over the desired ground surface. The detachable cap comprising of a plurality of pores holes allows the mixture to be extracted evenly from the seed dispenser 10. With the appropriate ratio of powdered indicator and groundcover plant seeds, only a light dusting of the ground surface intended for planting is required.

Traditional methods of spreading the minuscule Irish moss seeds without the powdered indicator lead to waste from over seeding or uneven distribution thus resulting is patched germination. The mixture of Irish moss seeds and powdered lime allows the planter to see the surface area that has already been seeded. The powdered lime not only acts as a visual indicator, it also allows the Irish moss seeds to be dispensed evenly without over clumping. The consistency and weight of the powdered lime mixed with the Irish moss seeds provide a fluidized mixture that overcomes prior issues where the smaller seeds by themselves were too minute in volume and weight to properly dispense.

In alternative embodiments, the seed mixtures ratio is dependent on the type of groundcover plant and its relative seed size. For example, ice plant seeds, ranging from approximately 12,000 to 20,000 seeds per gram, are mixed with powdered lime to once again provide for easy application. The operable mixture ratio is 96-99.99 percent by total volume of the powdered lime mixed with 0.01-4 percent by total volume ice plant seeds. The preferred mixture ratio is 98-99.99 percent by total volume of powdered lime mixed with 0.01-2 percent by total volume of ice plant seeds. The optimal mixture ration is 99-99.99 percent powdered lime mixed with 0.01-1 percent by total volume ice plant seeds.

Table 1 shows the different groundcover plant species, their relative seed size, and the operable, preferred and optimal mixture ratio between the indicated groundcover plant seeds and the powdered indicator.

TABLE 1

| | | Operable ratio | Preferred ratio | Optimal ratio |
|---|---|---|---|---|
| | | between groundcover | between groundcover | between groundcover |
| | | plant seeds and | plant seeds and | plant seeds and |
| | | powdered indicator | powdered indicator | powdered indicator |
| | Approximate seed | (percent by total | (percent by total | (percent by total |
| Groundcover plant species | size (seeds per gram) | volume) | volume) | volume) |
| Irish Moss, Chamomile, | 40,000-60,000 | 98-99.99% powdered | 99.-99.99% powdered | 99.51-99.99% |
| Lobelia | | indicator mixed with | indicator mixed with | powdered indicator |
| | | .01-2% groundcover | .01-1% groundcover | mixed with .01-.49% |
| | | plant seeds | plant seeds | groundcover plant |
| | | | | seeds |
| Ice Plant, Sedum, Saxifraga, | 12,000-20,000 | 96-99.99% powdered | 98.-99.99% powdered | 99.01-99.99% |
| Creeping Speedwell, Moss | | indicator mixed with | indicator mixed with | powdered indicator |
| Rose | | .01-4% groundcover | .01-2% groundcover | mixed with .01-.99% |
| | | plant seeds | plant seeds | groundcover plant |
| | | | | seeds |
| Creeping Thyme, Aubrieta, | 4,000-8,000 | 94-99.99% powdered | 96-99.99% powdered | 98-99% powdered |
| Cymbalaria, Leontopodium | | indicator mixed with | indicator mixed with | indicator mixed with |
| | | .01-6% groundcover | .01-4% groundcover | 1-2% groundcover |
| | | plant seeds | plant seeds | plant seeds |
| Cerastium, Alyssum, | 1000-4000 | 90-99.99% powdered | 94-99.99% powdered | 97-99% powdered |
| Prunella, Erigeron | | indicator mixed with | indicator mixed with | indicator mixed with |
| | | .01-10% groundcover | .01-6% groundcover | 1-3% groundcover |
| | | plant seeds | plant seeds | plant seeds |

Throughout the specification the aim has been to describe the invention without limiting the invention to any one embodiment or specific collection of features. Persons skilled in the relevant art may realize variations from the specific embodiment that will nonetheless fall within the scope of the invention. For example, the seed dispenser may be of any type known in the art. Any traditional seed dispensers may be used to spread the properly mixed ratio of powder indicator and groundcover plant seeds.

The powdered indicator may be any powdered material that is capable of being evenly mixed with the small groundcover plant seeds and provides visible indication to allow planters to see the location of prior applications. While the preferred powered lime has the benefit of soil ameliorative attributes, the powdered indicator need not have any fertilization attributes and may comprise of powdered flower or talcum powder.

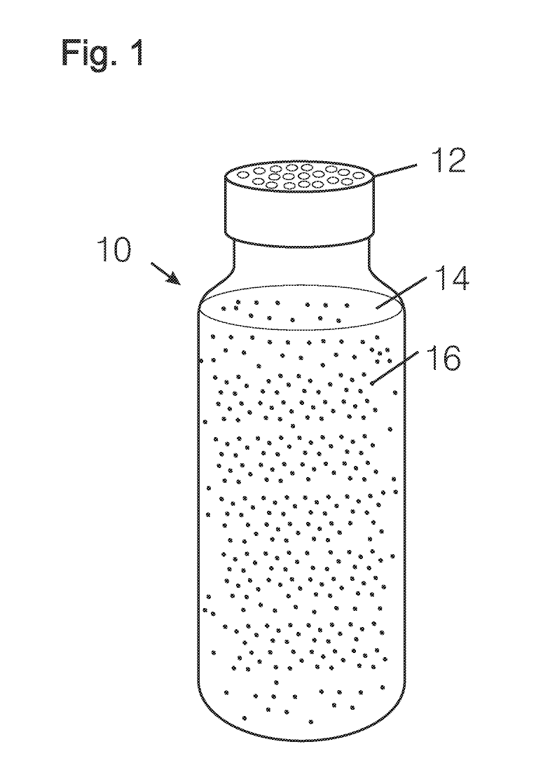

The groundcover plant seeds may comprise of any single species or a mixture of multiple species of groundcover plants. FIG. 1 is a general depiction and is not meant to represent an exact mixture ratio nor any particular size or specie of groundcover plant seed.

Accordingly, the scope of the invention should be determined not by the embodiment illustrated, but by the appended claims and their legal equivalents.

I claim:

1. A groundcover plant seed mixture comprising a majority percentage by total volume of a powdered indicator and a minority percentage by total volume of groundcover plant seeds.

2. The groundcover plant seed mixture of claim 1 wherein the said majority percentage by total value of said powdered indicator range from 90 to 99.99 percent and the said minority percentage by total volume of said groundcover plant seeds range from 0.01 to 10 percent.

3. The groundcover plant seed mixture of claim 1 wherein the said groundcover seeds germinate at least one species of groundcover plant comprising: chamomile, lobelia, ice plant, sedum, saxifraga, creeping speedwell, moss rose, creeping thyme, aubrieta, cymbalaria, leontopodium, cerastium, alyssum, prunella, and erigeron.

4. The groundcover plant seed mixture of claim 1 wherein the said powdered indicator is comprised of powdered lime.

5. The groundcover plant seed mixture of claim 1 wherein the said groundcover plant seeds comprise Irish moss seeds.

6. The groundcover plant seed mixture of claim 1 wherein the size of said groundcover plant seeds range from 1,000-60,000 seeds per gram.

7. The groundcover seed mixture of claim 6 wherein the size of said groundcover plant seeds range from 1,000 to 4,000 seeds per gram.

8. The groundcover seed mixture of claim 6 wherein the size of said groundcover plant seeds range from 4,000 to 8,000 seeds per gram.

9. The groundcover seed mixture of claim 6 wherein the size of said groundcover plant seeds range from 12,000 to 20,000 seeds per gram.

10. The groundcover seed mixture of claim 6 wherein the size of said groundcover plant seeds range from 40,000 to 60,000 seeds per gram.

11. The groundcover seed mixture of claim 6 wherein an operable mixture ratio between said groundcover plant seeds sized between 40,000 to 60,000 is 98-99.99 percent by total volume of said powdered indicator mixed with 0.01 to 2 percent by total volume of said groundcover plant seeds.

12. The groundcover seed mixture of claim 6 wherein an operable mixture ratio between said groundcover plant seeds sized between 12,000 to 20,000 is 96-99.99 percent by total volume of said powdered indicator mixed with 0.01 to 4 percent by total volume of said groundcover plant seeds.

13. The groundcover seed mixture of claim 6 wherein an operable mixture ratio between said groundcover plant seeds sized between 4,001 to 8,000 is 94-99.99 percent by total volume of said powdered indicator mixed with 0.01 to 6 percent by total volume of said groundcover plant seeds.

14. The groundcover seed mixture of claim 6 wherein an operable mixture ratio between said groundcover plant seeds sized between 1,000 to 4,000 is 90-99.99 percent by total volume of said powdered indicator mixed with 0.01 to 10 percent by total volume of said groundcover plant seeds.

15. A method of dispensing groundcover plant seeds comprising
a. Mixing a majority percentage by total volume of a powdered indicator and a minority percentage by total volume percent by total volume of groundcover plant seeds inside a portable seed dispenser.
b. Shaking the said portable dispenser vigorously to allow the said powdered indicator to mix evenly with the said groundcover plant seeds.
c. Spreading the said groundcover seeds and said powdered indicator mixture by gently shaking the said portable seed dispenser and applying a light dusting of the said mixture over a desired planting area.

16. The method of dispensing groundcover plant seeds of claim 15 wherein said powdered indicator comprises powdered lime.

17. The method of dispensing groundcover plant seeds of claim 15 wherein the said groundcover plant seeds germinate of at least one species of groundcover plant comprising: Irish moss, chamomile, lobelia, ice plant, sedum, saxifraga, creeping speedwell, moss rose, creeping thyme, aubrieta, cymbalaria, leontopodium, cerastium, alyssum, prunella, and erigeron.

18. The method of dispensing groundcover plant seeds of claim 15 wherein the said majority percentage by total value of said powdered indicator range from 90 to 99.99 percent and the said minority percentage by total volume of said groundcover plant seeds range from 0.01 to 10 percent.

19. The method of dispensing groundcover plant seeds of claim 15 wherein the size of said groundcover plant seeds range from 1,000 to 60,000 seeds per gram.
